SEQUENCE LISTING (4 sequences)

SEQ ID NO: 1 - protein, 433 aa, Homo sapiens
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 2 - protein, 228 aa, Homo sapiens
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 3 - protein, 121 aa, Homo sapiens
SAASASAASDVSVESTAEATVCTVTLEKMSAGLGFSLEGGKGSLHGDKPLTINRIFKGAASEQSETVQPGDEILQLGGTAMQGLTRFEAWNIIKALPDGPVTIVIRRKSLQSKETTAAGDS

SEQ ID NO: 4 - protein, 846 aa, Homo sapiens
SLWGLVSKMPPEKVQRLYVDFPQHLRHLLGDWLESQPWEFLVGSDAFCCNLASALLSDTVQHLQASVGEQGEGSTILQHISTLESIYQRDPLKLVATFRQILQGEKKAVMEQFRHLPMPFHWKQEELKFKTGLRRLQHRVGEIHLLREALQKGAEAGQVSLHSLIETPANGTGPSEALAMLLQETTGELEAAKALVLKRIQIWKRQQQLAGNGAPFEESLAPLQERCESLVDIYSQLQQEVGAAGGELEPKTRASLTGRLDEVLRTLVTSCFLVEKQPPQVLKTQTKFQAGVRFLLGLRFLGAPAKPPLVRADMVTEKQARELSVPQGPGAGAESTGEIINNTVPLENSIPGNCCSALFKNLLLKKIKRCERKGTESVTEEKCAVLFSASFTLGPGKLPIQLQALSLPLVVIVHGNQDNNAKATILWDNAFSEMDRVPFVVAERVPWEKMCETLNLKFMAEVGTNRGLLPEHFLFLAQKIFNDNSLSMEAFQHRSVSWSQFNKEILLGRGFTFWQWFDGVLDLTKRCLRSYWSDRLIIGFISKQYVTSLLLNEPDGTFLLRFSDSEIGGITIAHVIRGQDGSPQIENIQPFSAKDLSIRSLGDRIRDLAQLKNLYPKKPKDEAFRSHYKPEQMGKDGRGYVPATIKMTVERDQPLPTPELQMPTMVPSYDLGMAPDSSMSMQLGPDMVPQVYPPHSHSIPPYQGLSPEESVNVLSAFQEPHLQMPPSLGQMSLPFDQPHPQGLLPCQPQEHAVSSPDPLLCSDVTMVEDSCLSQPVTAFPQGTWIGEDIFPPLLPPTEQDLTKLLLEGQGESGGGSLGAQPLLQPSHYGQSGISMSHMDLRANPSW

(19) United States
(12) Patent Application Publication
(10) Pub. No.: US 2009/0130661 A1
(43) Pub. Date: May 21, 2009

(54) Method for Detecting IL-16 Activity and Modulation of IL-16 Activity Based on Phosphorylated Stat-6 Proxy Levels

(76) Inventors: William G. Glass, Libertyville, IL (US); Changbao Liu, Radnor, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/860,083

(22) Filed: Sep. 24, 2007

Related U.S. Application Data
(60) Provisional application No. 60/827,313, filed on Sep. 28, 2006.

(51) Int. Cl. C12Q 1/68

(52) U.S. Cl. 435/6

(57) ABSTRACT

Methods for detecting IL-16 biological activity, detecting modulation of IL-16 biological activity, and diagnosing the presence of, or susceptibility to, an IL-16-related disorder in a subject involve measuring and comparing the levels of a phosphorylated STAT-6 proxy produced by eukaryotic cells expressing CD4 or CD9, peripheral blood mononuclear cells, HuT-78 cells, or THP-1 cells.

### CLAIM TO PRIORITY

This application claims the benefit of U.S. Provisional Application Ser. No. 60/827,313, filed 28 Sep. 2006, the entire disclosure of which is incorporated herein by reference in its entirety.

### FIELD OF THE INVENTION

The present invention relates to a method for detecting IL-16 biological activity and detecting modulation of IL-16 biological activity. Additionally, the present invention is directed to a method of diagnosing the presence of, or susceptibility to, an IL-16 related disorder in a subject.

### BACKGROUND OF THE INVENTION

Interleukin-16 (IL-16; SEQ ID NO: 3) is a pro-inflammatory cytokine that induces positive chemotaxis of T-lymphocytes, monocytes, eosinophils, and dendritic cells (67 J. Leukocyte Biol. 757 (2000)). IL-16 stimulus also increases IL-1b expression, increases IL-6 expression, and increases IL-15 expression in IL-16 responsive eukaryotic cells (67 J. Leukocyte Biol. 757 (2000)).

IL-16 peptide chain monomers are formed by the caspase-3 mediated proteolytic processing of a larger 14 kDa precursor molecule (273 J. Biol. Chem. 1144 (1998)). IL-16 monomers form tetrameric peptide chain complexes. These tetrameric IL-16 complexes are believed to be the bioactive form of IL-16 (67 J. Leukocyte Biol. 757 (2000)). Eukaryotic cells that produce IL-16 include cells that express CD4 or CD8, such as T-cells, mast cells, eosinophils, dendritic cells epithelial cells, fibroblasts, and cells of the cerebellum (67 J. Leukocyte Biol. 757 (2000)). Eukaryotic cells responsive to IL-16 express the CD4 and CD9 peptide chains, but the response to IL-16 may also be independent of these peptide chains (see e.g. 164 J. Immunol. 4429 (2000)).

IL-16 has been reported to play an important role in such diseases as asthma, atopic dermatitis, and rheumatoid arthritis, among others (see e.g. 162 Am. J. Respir. Crit. Care Med. 105 (2000); 109 J. Allergy Clin. Immunol. 681 (2002); 31 J. Rheumatol. 35 (2004). For example, in human patients IL-16 has been shown to be responsible for attracting asthma inducing cells to the lungs and to play a critical role in triggering asthmatic responses in patients (162 μm. J. Respir. Crit. Care Med. 105 (2000)). Clearly, the ability to detect and identify molecules that activate or inhibit IL-16 is critical to the development of effective treatments for IL-16 mediated diseases.

Thus, a need exists for novel methods for detecting IL-16 biological activity, activators of IL-16 biological activity, inhibitors of IL-16 biological activity, and identifying individuals with IL-16 related disorders.

### BRIEF DESCRIPTION OF THE DRAWINGS

FIG. 1 is a graph showing that IL-16 biological activity increases phosphorylated STAT-6 (P-STAT) levels in peripheral blood mononuclear cells (PBMCs) relative to controls.

FIG. 2 is a graph showing that IL-16 biological activity increases phosphorylated STAT-6 levels in THP-1 cells relative to controls.

FIG. 3 is a graph showing that inhibitors of IL-16 biological activity decrease phosphorylated STAT-6 levels in PBMC cells relative to controls.

FIG. 4 is a graph showing that inhibitors of IL-16 biological activity decrease phosphorylated STAT-6 levels in THP-1 cells relative to controls.

### SUMMARY OF THE INVENTION

One aspect of the invention is a method of detecting IL-16 biological activity in a sample comprising the steps of providing a first population of eukaryotic cells surrounded by media and responsive to IL-16 biological activity with a first test sample; providing a second population of eukaryotic cells surrounded by media and responsive to IL-16 biological activity with a negative control sample; measuring the amount of a phosphorylated STAT-6 proxy produced by the first and second populations of eukaryotic cells; and comparing the amount of a phosphorylated STAT-6 proxy produced by the first and second populations of eukaryotic cells, wherein a larger amount of a phosphorylated STAT-6 proxy produced by the first population of eukaryotic cells relative to the phosphorylated STAT-6 proxy level produced by the second population of eukaryotic cells indicates the detection of IL-16 biological activity in the test sample.

Another aspect of the invention is a method of detecting a molecule that increases IL-16 biological activity in a sample comprising the steps of providing a first population of eukaryotic cells surrounded by media and responsive to IL-16 biological activity with a first test sample; providing a second population of eukaryotic cells surrounded by media and responsive to IL-16 biological activity with a positive control sample containing biologically active IL-16; measuring the amount of a phosphorylated STAT-6 proxy produced by the first and second populations of eukaryotic cells; and comparing the amount of a phosphorylated STAT-6 proxy produced by the first and second populations of eukaryotic cells, wherein a larger amount of a phosphorylated STAT-6 proxy produced by the first population of eukaryotic cells relative to the phosphorylated STAT-6 proxy level produced by the second population of eukaryotic cells indicates the presence of a molecule that increases IL-16 biological activity in the test sample.

Another aspect of the invention is a method of detecting a molecule that decreases IL-16 biological activity in a sample comprising the steps of providing a first population of eukaryotic cells surrounded by media and responsive to IL-16 biological activity with a first test sample; providing a second population of eukaryotic cells surrounded by media and responsive to IL-16 biological activity with a positive control sample containing biologically active IL-16; measuring the amount of a phosphorylated STAT-6 proxy produced by the first and second populations of eukaryotic cells; and comparing the amount of a phosphorylated STAT-6 proxy produced by the first and second populations of eukaryotic cells, wherein a smaller amount of a phosphorylated STAT-6 proxy produced by the first population of eukaryotic cells relative to the phosphorylated STAT-6 proxy level produced by the second population of eukaryotic cells indicates the presence of a molecule that decreases IL-16 biological activity in the test sample.

Another aspect of the invention is a method of diagnosing the presence of, or susceptibility to, an IL-16-related disorder in a first subject comprising the steps of providing a first population of eukaryotic cells from a first subject; providing a second population of eukaryotic cells comprising cells selected from the group consisting of cells from a second subject not susceptible to an IL-16 related disorder, cells from a second subject not afflicted with an IL-16 related disorder, and cells not characterized by increased IL-16 expression or increased IL-16 biological activity; measuring the amount of a phosphorylated STAT-6 proxy in the first and second populations of eukaryotic cells; and comparing the amount of a phosphorylated STAT-6 proxy in the first and second populations of eukaryotic cells wherein a larger amount of a phosphorylated STAT-6 proxy in the first population of eukaryotic cells relative to the phosphorylated STAT-6 proxy level in the second population of eukaryotic cells indicates the presence of, or susceptibility to, an IL-16-related disorder.

### DETAILED DESCRIPTION OF THE INVENTION

All publications, including but not limited to patents and patent applications, cited in this specification are herein incorporated by reference as though fully set forth.

As used herein and in the claims, the singular forms “a,” “and,” and “the” include plural reference unless the context clearly dictates otherwise. Thus, for example, reference to “a cell” is a reference to one or more cells and includes equivalents thereof known to those skilled in the art.

Unless defined otherwise, all technical and scientific terms used herein have the same meaning as commonly understood by one of ordinary skill in the art to which this invention belongs. Although any compositions and methods similar or equivalent to those described herein can be used in the practice or testing of the invention, exemplary compositions and methods are described herein.

The term “antibody” means immunoglobulin or antibody molecules comprising polyclonal antibodies, monoclonal antibodies including murine, human, humanized and chimeric monoclonal antibodies and antibody fragments, portions, or variants, including, without limitation, single chain antibodies, single domain antibodies, monovalent and multivalent antibodies, and the like. Antibodies are secreted proteins constitutively expressed and secreted by plasma cells. Antibodies can also be produced using plasma cells immortalized by standard methods such as hybridoma generation or by transfection of antibody heavy and/or light chain genes into an immortalized B cell such as a myeloma cell or other cell types, such as Chinese hamster ovary (CHO) cells, plant cells and insect cells.

The term “biological activity” means the response of a biological system to a molecule. Such biological systems may be, for example, a cell, a replicable nucleic acid, such as a virus or plasmid, the isolated components of a cell or replicable nucleic acid, or an in vitro system incorporating one or more of these.

The term “CD4” means a peptide chain with at least 50% identity to residues 1 to 433 of SEQ ID NO: 1 and that is responsive to IL-16. Identity between two peptide chains can be determined by pair-wise amino acid sequence alignment using the default settings of the AlignX module of Vector NTI v.9.0.0 (Invitrogen Corp., Carslbad, Calif.). AlignX uses the CLUSTALW algorithm to perform pair-wise amino acid sequence alignments. “CD4” is an acryonym for “Cluster of Determinant antigen 4.”

The term “CD9” means a peptide chain with at least 90% identity to residues 1 to 228 of SEQ ID NO: 2 and that is responsive to IL-16. “CD9” is an acryonym for “Cluster of Determinant antigen 9.”

The term “eukaryotic cell” means a cell in which genetic material is organized into at least one membrane-bound nucleus.

The term “express” means the detectable production of a peptide chain encoded by a nucleic acid.

The term “IL-16” means a peptide chain with at least 80% identity to amino acid residues 1 to 121 of SEQ ID NO: 3 that can bind CD4 and increase production of a phosphorylated STAT-6 proxy. “IL-16” is an acronym for “Interleukin 16.”

The term “IL-16-related disorder” means an infectious or immune mediated inflammatory disorder, such as tuberculosis, pneumonia, respiratory syncytial virus, asthma, atopic dermatitis, Crohn's disease, inflammatory bowel disease, rheumatoid arthritis, central nervous system related disorders, such as multiple sclerosis, systemic lupus erythematosis, Graves disease, hepatitis C virus, mumps, coxsackie, echovirus, influenza, E. Coli infection, listeria, meningitis, Epstein-Barr virus, and related diseases and disorders characterized by increased IL-16 biological activity.

The term “peptide chain” means a molecule that comprises at least two amino acid residues linked by a peptide bond to form a chain. Large peptide chains of more than 50 amino acids may be referred to as “polypeptides” or “proteins.” Small peptide chains of less than 50 amino acids may be referred to as “peptides.”

The term “HuT-78 cells” means cells with ATCC® Number: TIB-161™ from the American Type Culture Collection (ATCC), Manassas, Va. or cells derived from these.

The term “THP-1 cells” means cells with ATCC® Number: TIB-202™ from the American Type Culture Collection (ATCC), Manassas, Va. or cells derived from these.

The term “population” means at least two items such as two cells.

The term “phosphorylated STAT-6 proxy,” means a phosphorylated peptide chain with at least 80% identity to amino acid residues 1 to 846 of SEQ ID NO: 4, a peptide chain expressed by activating the regulatory region of a gene responsive to a phosphorylated peptide chain with at least 80% identity to amino acid residues 1 to 846 of SEQ ID NO: 4, or a nucleic acid transcribed by activating the regulatory region of a gene responsive to a phosphorylated peptide chain with at least 80% identity to amino acid residues 1 to 846 of SEQ ID NO: 4. A phosphorylated STAT-6 proxy can be used as an indicator of STAT-6 peptide chain activation. Identity between two peptide chains can be determined by pair-wise amino acid sequence alignment using the default settings of the AlignX module of Vector NTI v.9.0.0 (Invitrogen Corp., Carslbad, Calif.). AlignX uses the CLUSTALW algorithm to perform pair-wise amino acid sequence alignments. “STAT” is an acronym for “signal transducers and activators of transcription.” STAT-6 is an intracellular peptide chain that is phosphorylated, typically on a tyrosine residue, in response to signaling by interleukins such as IL-3, IL-4, and IL-13. Phosphorylation of STAT-6 activates STAT-6. Activated STAT-6 induces transcription of interleukin responsive genes by binding discrete response elements in DNAs. Regulatory regions in nucleic acids such as DNAs may comprise, or consist of, such discrete response elements. Activated STAT-6 induces, for example, transcription of the genes encoding Homo sapiens BCL2-like 1 isoform 2 and BCL-X(L).

The term “responsive” means capable of producing a detectable signal in reaction to a stimulus.

One aspect of the invention is a method of detecting IL-16 biological activity in a sample comprising the steps of providing a first population of eukaryotic cells surrounded by media and responsive to IL-16 biological activity with a first test sample; providing a second population of eukaryotic cells surrounded by media and responsive to IL-16 biological activity with a negative control sample; measuring the amount of a phosphorylated STAT-6 proxy produced by the first and second populations of eukaryotic cells; and comparing the amount of a phosphorylated STAT-6 proxy produced by the first and second populations of eukaryotic cells, wherein a larger amount of a phosphorylated STAT-6 proxy produced by the first population of eukaryotic cells relative to the phosphorylated STAT-6 proxy level produced by the second population of eukaryotic cells indicates the detection of IL-16 biological activity in the test sample.

One of ordinary skill in the art will readily be able to appreciate the increase or decrease in STAT-6 proxy (e.g., fold change) that would satisfy the invention by doing routine statistical analysis of the data, i.e., data points analyzed for cell types tested compared to reference standards, etc. and by confirmatory analysis of IL-16 activity. For example, IL-16 activity can be measured by ELISA assay, measuring IL-4 secretion, chemotaxis, and the like.

Eukaryotic cells useful in the methods of the invention may be adherent or in suspension. These eukaryotic cells may be surrounded by media suitable for cell growth or maintenance that contains serum or is serum free. Eukaryotic cells useful in the methods of the invention are responsive to IL-16. IL-16 responsive cells respond to IL-16 stimulus by chemotaxis toward an IL-16 source, increased IL-1b expression, increased IL-6, increased IL-15 expression, decreased RANTES production, or increased phosphorylated STAT-6 production and can be identified on these bases. Cells that are responsive to IL-16 typically express CD4, CD9 or both CD4 and CD9 any may also be identified on this basis. Test samples and negative control samples may comprise a carrier that is compatible with maintaining IL-16 biological activity in a sample and is compatible with the eukaryotic cells used in the methods of the invention. Phosphate buffered saline (PBS) is one example of such a carrier, those skilled in the art will recognize others. Ideally, negative control samples are known to contain no detectable IL-16 biological activity.

Phosphorylated STAT-6 proxy production may be measured in a variety of different ways. For example, where the phosphorylated STAT-6 proxy is a peptide chain, production can be measured by phosphorylated STAT-6 proxy expression assays that specifically detect phosphorylated STAT-6 proxy peptide chains. Such assays may include SDS-PAGE, Western blotting, ELISA, phosphorylated STAT-6 proxy specific enzyme assays such as luciferase assays, or phosphorylated STAT-6 proxy specific antibody conjugated bead analyses. Such phosphorylated STAT-6 proxy peptide chains may be the STAT-6 peptide chain of SEQ ID NO: 4 or a peptide chain expressed by activating the regulatory region of a gene responsive to a phosphorylated peptide chain with at least 80% identity to amino acid residues 1 to 846 of SEQ ID NO: 4. The peptide chain encoded by a nucleic acid sequence under the control of the regulatory region of a gene responsive to a phosphorylated peptide chain with at least 80% identity to amino acid residues 1 to 846 of SEQ ID NO: 4 may be an easily detected peptide such as, for example, luciferase or green fluorescent protein. Those skilled in the art will recognize other easily detected peptide chains suitable for use in the methods of the invention. Alternatively, where the phosphorylated STAT-6 proxy is an RNA its production can be measured by RT-PCR, Northern blotting, or other techniques well known by those skilled in the art for detecting specific RNA transcripts.

A nucleic acid sequence may be placed under the control of the regulatory region of a gene responsive to a phosphorylated peptide chain with at least 80% identity to amino acid residues 1 to 846 of SEQ ID NO: 4 by operably linking this regulatory region to the nucleic acid sequence. Such an operable linkage may be created in the context of an extra-chromosomal nucleic acid, such as a plasmid, that can be used as an extra-chromosomal reporter construct encoding a peptide chain or RNA. Such extra-chromosomal constructs may also be introduced into the chromosomal DNA by random recombination events using transfection techniques well known in the art. Alternatively, such operable linkages may be created in the context of a chromosomal nucleic acid such as chromosomal DNA. The chromosomal DNA genes encoding the Homo sapiens BCL2-like 1 isoform 2 peptide chain or BCL-X(L) peptide chain are examples of such operable linkages in the context of a chromosomal nucleic acid. The chromosomal DNAs encoding the genes for the BCL2-like 1 isoform 2 peptide chain or BCL-X(L) peptide chain are under the control of a regulatory element responsive to phosphorylated STAT-6. However, as those skilled in the art will recognize, site-specific recombination techniques can be used to operably link a heterologous gene to the regulatory region of a native gene present in chromosomal DNA. The resulting peptide chain or RNA encoded by such a chromosomal nucleic acid can then function as a phosphorylated STAT-6 proxy.

In one embodiment of the method the eukaryotic cells express a CD4 peptide chain or CD9 peptide chain. CD4 or CD9 peptide chains may be constitutively or inducibly expressed and may be encoded by native genes or heterologous nucleic acids such as cDNAs. Such cDNAs may, for example, encode the peptide chain of SEQ ID NO: 1 or SEQ ID NO: 2.

In another embodiment of the method the eukaryotic cells are selected from the group consisting of peripheral blood mononuclear cells, HuT-78 cells, and THP-1 cells. These eukaryotic cells may be surrounded by media suitable for cell growth or maintenance that contains serum or is serum free.

In another embodiment of the method providing the first test sample produces a final IL-16 concentration in the media surrounding the first population of eukaryotic cells that is 100 ng/ml to 5000 ng/ml. The IL-16 assay methods described here are capable of detecting IL-16 biological activity present in the media surrounding the eukaryotic cells at a concentration of at least 100 ng/ml to 5000 ng/ml IL-16. However, the methods of the invention are also suitable for detecting higher and lower final concentrations of IL-16 in a sample that are outside this range.

In another embodiment of the method the phosphorylated STAT-6 is intracellular. The peptide chain comprising the amino acid sequence of SEQ ID NO: 4 is an example of a phosphorylated STAT-6 proxy that is intracellular. Such phosphorylated STAT-6 proxies may also be generated by expressing a fusion peptide chain comprising a nuclear localization signal sequence, or other non-secretory signal sequence, fused to a phosphorylated STAT-6 proxy peptide chain. Those skilled in the art will recognize other appropriate signal sequences which function to limit extracellular secretion of a peptide chain.

Another aspect of the invention is a method of detecting a molecule that increases IL-16 biological activity in a sample comprising the steps of providing a first population of eukaryotic cells surrounded by media and responsive to IL-16 biological activity with a first test sample; providing a second population of eukaryotic cells surrounded by media and responsive to IL-16 biological activity with a positive control sample containing biologically active IL-16; measuring the amount of a phosphorylated STAT-6 proxy produced by the first and second populations of eukaryotic cells; and comparing the amount of a phosphorylated STAT-6 proxy produced by the first and second populations of eukaryotic cells, wherein a larger amount of a phosphorylated STAT-6 proxy produced by the first population of eukaryotic cells relative to the phosphorylated STAT-6 proxy level produced by the second population of eukaryotic cells indicates the presence of a molecule that increases IL-16 biological activity in the test sample. This method of the invention may be used to detect or identify molecules such as drugs that increase IL-16 biological activity. Such molecules may increase IL-16 biological activity by any mechanism.

Positive control samples may comprise a carrier that is compatible with maintaining IL-16 biological activity in a sample and is compatible with the eukaryotic cells used in the methods of the invention. Phosphate buffered saline (PBS) is one example of such a carrier, those skilled in the art will recognize others. Positive control samples are known to contain detectable IL-16 biological activity.

In one embodiment of the method the eukaryotic cells express a CD4 peptide chain or CD9 peptide chain.

In another embodiment of the method the eukaryotic cells are selected from the group consisting of peripheral blood mononuclear cells, HuT-78 cells, and THP-1 cells.

In another embodiment of the method the first test sample comprises an antibody molecule.

In another embodiment of the method the phosphorylated STAT-6 proxy is intracellular.

Another aspect of the invention is a method of detecting a molecule that decreases IL-16 biological activity in a sample comprising the steps of providing a first population of eukaryotic cells surrounded by media and responsive to IL-16 biological activity with a first test sample; providing a second population of eukaryotic cells surrounded by media and responsive to IL-16 biological activity with a positive control sample containing biologically active IL-16; measuring the amount of a phosphorylated STAT-6 proxy produced by the first and second populations of eukaryotic cells; and comparing the amount of a phosphorylated STAT-6 proxy produced by the first and second populations of eukaryotic cells, wherein a smaller amount of a phosphorylated STAT-6 proxy produced by the first population of eukaryotic cells relative to the phosphorylated STAT-6 proxy level produced by the second population of eukaryotic cells indicates the presence of a molecule that decreases IL-16 biological activity in the test sample. This method of the invention may be used to detect or identify molecules such as drugs that inhibit IL-16 biological activity. Such molecules may inhibit IL-16 biological activity by any mechanism.

In one embodiment of the method the eukaryotic cells express a CD4 peptide chain or CD9 peptide chain.

In another embodiment of the method the eukaryotic cells are selected from the group consisting of peripheral blood mononuclear cells, HuT-78 cells, and THP-1 cells.

In another embodiment of the method the first test sample comprises an antibody molecule.

In another embodiment of the method the phosphorylated STAT-6 proxy is intracellular.

Another aspect of the invention is a method of diagnosing the presence of, or susceptibility to, an IL-16-related disorder in a first subject comprising the steps of providing a first population of eukaryotic cells from a first subject; providing a second population of eukaryotic cells comprising cells selected from the group consisting of cells from a second subject not susceptible to an IL-16 related disorder, cells from a second subject not afflicted with an IL-16 related disorder, and cells not characterized by increased IL-16 expression or increased IL-16 biological activity; measuring the amount of a phosphorylated STAT-6 proxy in the first and second populations of eukaryotic cells; and comparing the amount of a phosphorylated STAT-6 proxy in the first and second populations of eukaryotic cells wherein a larger amount of a phosphorylated STAT-6 proxy in the first population of eukaryotic cells relative to the phosphorylated STAT-6 proxy level in the second population of eukaryotic cells indicates the presence of, or susceptibility to, an IL-16-related disorder.

In one embodiment of the method the eukaryotic cells express a CD4 peptide chain or CD9 peptide chain.

In another embodiment of the method the eukaryotic cells are peripheral blood mononuclear cells.

The present invention is further described with reference to the following examples. These examples are merely to illustrate aspects of the present invention and are not intended as limitations of this invention.

### EXAMPLE 1

### Assay Method for Detecting IL-16 Activity and Positive or Negative Modulation of IL-16 Activity Based on Increased STAT-6 Phosphorylation Levels

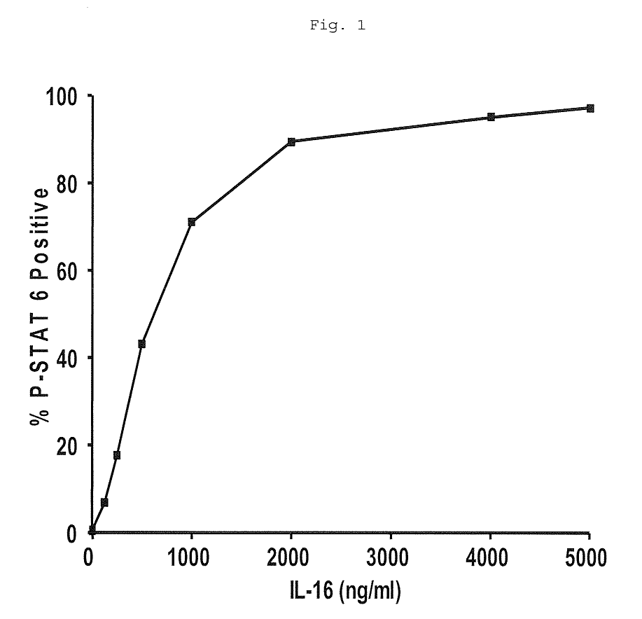

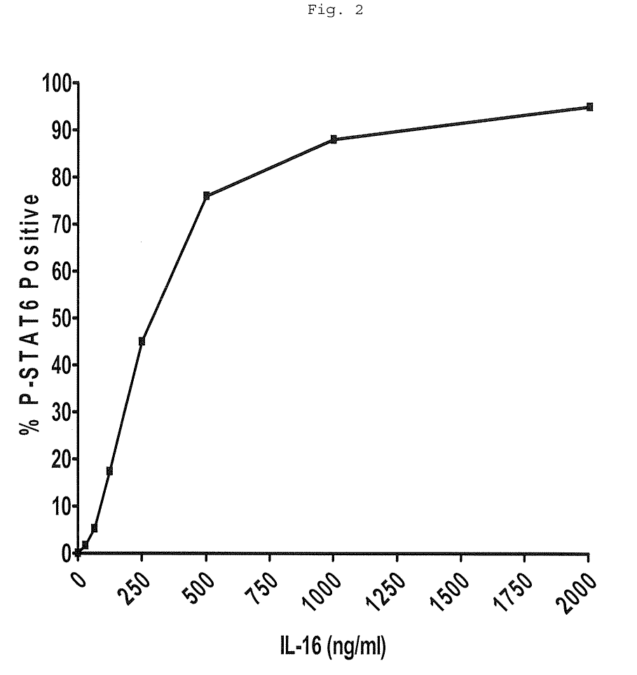

The presence of IL-16 biological activity in a test sample increases the level of phosphorylated STAT-6 produced by IL-16 responsive peripheral blood mononuclear cells (PBMCs) or eukaryotic cell lines relative to negative control cells unexposed to a sample containing biologically active IL-16 (FIGS. 1 and 2). Detection of increased levels of phosphorylated STAT-6 produced by PBMCs (FIG. 1) or IL-16 responsive eukaryotic THP-1 cells (FIG. 2) receiving a test sample containing unknown molecules can be used to assay for the presence of biologically active IL-16 in the test sample. This assay method can also be used to detect increased or decreased IL-16 biological activity in two different test samples (FIGS. 1 and 2).

Human PBMCs were isolated and maintained in serum free AIM-V® cell culture media (Invitrogen Inc., Carlsbad, Calif.) using standard methods (see e.g. 74(4) Blood. 1348 (1989)). Immortalized eukaryotic THP-1 human monocyte cells (ATCC® Number: TIB-202™; American Type Culture Collection (ATCC), Manassas, Va.) expressing CD4 (Cluster of Determinant antigen 4) and responsive to IL-16 activity were maintained using standard eukaryotic cell culture techniques in Roswell Park Memorial Institute 1640 (RPMI-1640; Invitrogen Inc., Carlsbad, Calif.) cell culture media containing 10% v/v Fetal Bovine Serum (FBS; Invitrogen Inc., Carlsbad, Calif.).

IL-16 biological activity in test samples was assayed using either isolated PBMC (FIG. 1) or THP-1 cells (FIG. 2). First, approximately 500,000 PBMC or THP-1 cells were placed in the wells of a 96 well tissue culture plate containing cell culture media (AIM-V or RPMI-1640) appropriate for either PBMC (FIG. 1) or THP-1 cell (FIG. 2) maintenance. Second, a test sample containing biologically active, recombinant Homo sapiens IL-16 (Invitrogen Corp., Carlsbad, Calif.) in phosphate buffered saline (PBS) vehicle was added to each tissue culture well such that the final concentration of biologically active IL-16 in the media surrounding the cells was between 31.2 ng/ml and 5000 ng/ml (FIG. 1 and FIG. 2). Negative control PBMC and THP-1 cells did not have test samples containing IL-16 added to the culture wells and instead negative control samples containing PBS vehicle alone were added to the media surrounding these cells (FIG. 1 and FIG. 2). Third, cells receiving test samples containing biologically active IL-16 and negative control cells were then separately incubated for 20 minutes at 37° C. under standard eukaryotic cell culture conditions. Fourth, phosphorylated STAT-6 levels in cells receiving test samples or negative control cells was measured in cell lysates or in permeabilized cells by FACs using a detectably labeled monoclonal antibody specific for phosphorylated STAT-6. The monoclonal antibody was the Clone 18 IgG2A monoclonal (Invitrogen, Carlsbad, Calif.) which was used as directed by the manufacturer. Fifth, phosphorylated STAT-6 levels in cell lysates or permeabilized cells that received test samples were compared to phosphorylated STAT-6 levels in negative control cells. Increased levels of phosphorylated STAT-6 in cells receiving test samples relative to negative control cells was indicative of a test sample containing biologically active IL-16 (FIGS. 1 and 2). Last, phosphorylated STAT-6 levels in cells receiving a first test sample containing biologically active IL-16 were compared to phosphorylated STAT-6 levels in cells that received a second test sample containing a different amount of biologically active IL-16. The test sample containing the highest amount of IL-16 biological activity can be identified by this comparison because this sample contains the highest level of phosphorylated STAT-6 in cells (FIGS. 1 and 2). The test sample containing the lowest amount of IL-16 biological activity can be identified by this comparison because this sample contains the lowest level of phosphorylated STAT-6 in the cell lysates or permeabilized cells (FIGS. 1 and 2).

These results demonstrate that IL-16 activity in a sample can be detected by a cell based assay method in which phosphorylated STAT-6 levels in IL-16 responsive cells exposed to a test sample containing biologically active IL-16 are increased relative to negative control cells that did not receive the test sample.

These results also demonstrate that the IL-16 biological activity assay described above can be used to identify test samples containing increased or decreased IL-16 activity. Consequently, the assay described above is suitable for the detection or identification of molecules that increase or decrease IL-16 activity in a sample. Such molecules may be drugs that increase IL-16 activity or simply be additional molecules of biologically active IL-16 that have been added to a sample. Alternatively, such molecules may be drugs that decrease IL-16 activity. Consequently, the assay described here can detect positive or negative modulation of IL-16 activity in a test sample and can be used to detect or identify molecules, such as drugs, that modulate IL-16 biological activity.

Data presented in FIG. 1 and FIG. 2 is representative of at least 3 independently conducted experiments.

### EXAMPLE 2

### Assay Method for Detecting Molecules that Modulate IL-16 Activity Based on Increased STAT-6 Phosphorylation Levels

The assay method described in Example 1 above may be modified to permit the detection or identification of molecules (e.g., drugs) in a test sample that modulate IL-16 activity. Such molecules may be, for example, inhibitors of IL-16 activity, for example, IL-16 antibodies and small molecules.

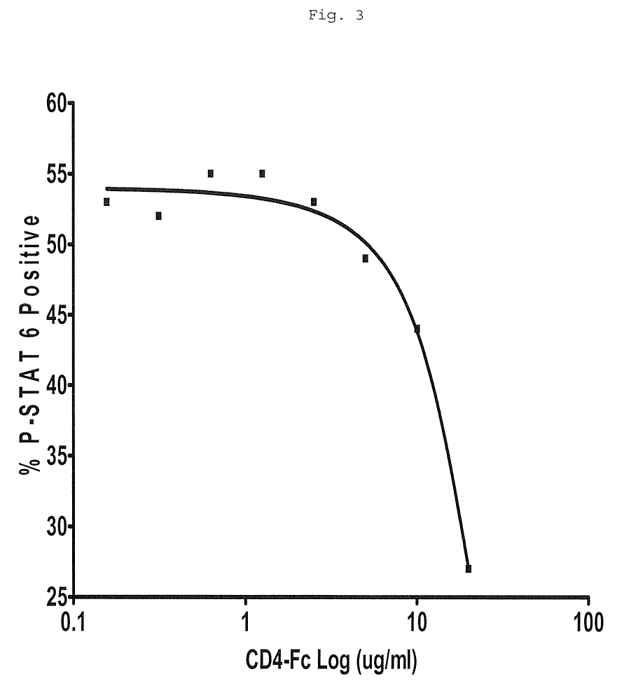

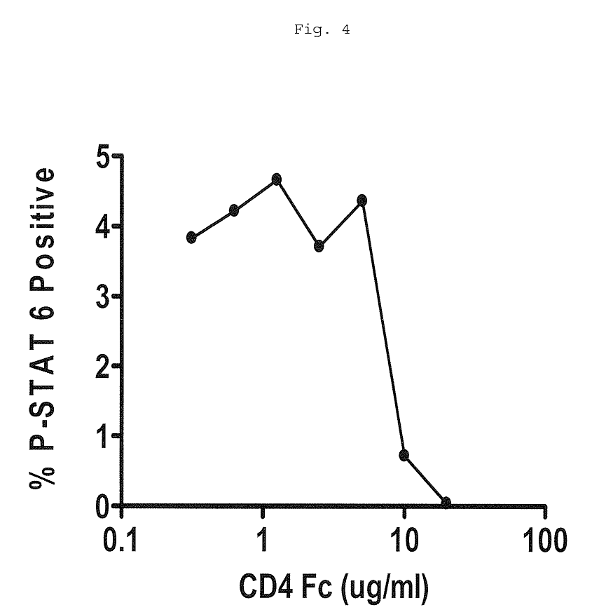

As shown in FIG. 3 and FIG. 4, STAT-6 phosphorylation levels in PBMC cells (FIG. 3) or THP-1 cells (FIG. 4) receiving a test sample that contains a first amount of an inhibitor of IL-16 biological activity is decreased relative to control cells receiving a test sample containing IL-16 and, second, a comparatively smaller amount of an inhibitor of IL-16. Consequently, the modified assay method described here can be used to detect or identify molecules that modulate IL-16 biological activity.

Assay methods for detecting or identifying molecules that modulate IL-16 activity were performed as follows. First, approximately 500,000 PBMC cells (FIG. 3) or THP-1 cells (FIG. 4) were placed in the wells of a 96 well tissue culture plate containing RPMI-1640 or D-MEM cell culture media as appropriate. PBMC cells and THP-1 cells were obtained and maintained as described in Example 1 above. Second, a test sample containing biologically active, recombinant Homo sapiens IL-16 (Invitrogen Corp., Carlsbad, Calif.) and a soluble CD4-Fc fusion protein that inhibits IL-16 biological activity in vitro was added to each tissue culture well. As indicated in FIG. 3 and FIG. 4, IL-16 was added such that the final concentration of biologically active IL-16 in the media surrounding the cells was either 500 ng/ml for PBMC cells or 1.25 μg/ml for THP-1 cells and the final concentration of the CD4-Fc fusion protein in each tissue culture well was between 156 ng/ml or 20,000 ng/ml.

CD4 is a receptor for IL-16. The soluble CD4-Fc fusion protein binds IL-16 in solution and modulates IL-16 activity by preventing IL-16 from activating CD4 receptors expressed on cells. The CD4-Fc fusion protein comprises a soluble CD4 protein sequence fused to an antibody Fc domain.

Additionally, the CD4-Fc fusion protein and IL-16 are mixed together in a phosphate buffered saline (PBS) vehicle for 30 minutes prior to addition to each tissue culture well. Negative control PBMC cells or THP-1 cells will not have IL-16 added to the culture wells and instead negative control samples containing PBS vehicle alone or a molecule that inhibits IL-16 activity will be added to the tissue culture wells. IL-16 modulators, such as IL-16 activity inhibitors, will be added to produce final modulator concentrations in the media surrounding the cells at which modulation of IL-16 activity is detectable. Positive control PBMC cells or THP-1 cells will receive biologically active IL-16 alone such that the final concentration of biologically active IL-16 in the media surrounding cells is sufficient to activate CD4 or other IL-16 receptors expressed by cells. Third, PBMC cells or THP-1 cells receiving test samples, negative control PBMC cells or THP-1 cells, and positive control PMBC cells or THP-1 cells will then be separately incubated for 20 minutes at 37° C. under standard eukaryotic cell culture conditions. Fourth, phosphorylated STAT-6 levels in PBMC cells or THP-1 cells receiving test samples, negative control PMBC cells or THP-1 cells, or positive control PBMC cells or THP-1 cells will be measured in cell lysates or in permeabilized cells by FACs using a detectably labeled monoclonal antibody specific for phosphorylated STAT-6. The monoclonal antibody is the Clone 18 IgG2A monoclonal (Invitrogen, Carlsbad, Calif.) which is used as directed by the manufacturer. Fifth, phosphorylated STAT-6 levels in cells that receive test samples containing biologically active IL-16 and the inhibitory CD4-Fc fusion protein will be compared to phosphorylated STAT-6 levels in positive control cells receiving biologically active IL-16 alone. Decreased levels of phosphorylated STAT-6 in cells receiving test samples relative to positive control cells will be indicative of a test sample containing an inhibitor of biologically active IL-16.

The foregoing demonstrates that the IL-16 biological activity assay described above can be used to detect or identify molecules that modulate IL-16 activity in a test sample (FIG. 3 and FIG. 4). Such molecules may be drugs that inhibit IL-16 activity or alternatively drugs that increase IL-16 activity. Data presented in FIG. 3 and FIG. 4 is representative of at least 3 independently conducted experiments.

The present invention now being fully described, it will be apparent to one of ordinary skill in the art that many changes and modifications can be made thereto without departing from the spirit or scope of the appended claims.

1. A method of detecting IL-16 biological activity in a sample, comprising the steps of:
a) providing a first population of eukaryotic cells surrounded by media and responsive to IL-16 biological activity in a first test sample;
b) measuring the amount of a phosphorylated STAT-6 proxy produced by the first population of eukaryotic cells; and
c) comparing the amount of a phosphorylated STAT-6 proxy produced by the first population of eukaryotic cells to an amount of a phosphorylated STAT-6 proxy produced by a negative control sample, wherein a larger amount of a phosphorylated STAT-6 proxy produced by the first population of eukaryotic cells relative to the phosphorylated STAT-6 proxy level produced by the negative control sample indicates the detection of IL-16 biological activity in the test sample.

2. The method of claim 1, wherein the providing step further comprises providing a second population of eukaryotic cells surrounded by media and responsive to IL-16 biological activity to form the negative control sample.

3. The method of claim 2, wherein the measuring step further comprises measuring the amount of a phosphorylated STAT-6 proxy produced by the second population of eukaryotic cells.

4. The method of claim 1, wherein the negative control sample is a second population of eukaryotic cells.

5. The method of claim 1, wherein the eukaryotic cells express a CD4 peptide chain or CD9 peptide chain.

6. The method of claim 5, wherein the eukaryotic cells are selected from the group consisting of peripheral blood mononuclear cells, HuT-78 cells, and THP-1 cells.

7. The method of claim 1, wherein providing the first test sample produces a final IL-16 concentration in the media surrounding the first population of eukaryotic cells that is 100 ng/ml to 5000 ng/ml.

8. The method of claim 1, wherein the phosphorylated STAT-6 proxy is intracellular.

9. A method of detecting a molecule that increases IL-16 biological activity in a sample comprising the steps of:
a) providing a first population of eukaryotic cells surrounded by media and responsive to IL-16 biological activity in a first test sample;
b) measuring the amount of a phosphorylated STAT-6 proxy produced by the first population of eukaryotic cells; and
c) comparing the amount of a phosphorylated STAT-6 proxy produced by the first population of eukaryotic cells with the amount of a phosphorylated STAT-6 proxy produced by a positive control sample containing biologically active IL-16, wherein a larger amount of a phosphorylated STAT-6 proxy produced by the first population of eukaryotic cells relative to the phosphorylated STAT-6 proxy level produced by the positive control sample indicates the presence of a molecule that increases IL-16 biological activity in the test sample.

10. The method of claim 9, wherein the providing step further comprises providing a second population of eukaryotic cells surrounded by media and responsive to IL-16 biological activity to form a positive control sample containing biologically active IL-16.

11. The method of claim 10, wherein the measuring step further comprises measuring the amount of a phosphorylated STAT-6 proxy produced by the second population of eukaryotic cells.

12. The method of claim 9, wherein the positive control sample is a second population of eukaryotic cells.

13. The method of claim 9, wherein the eukaryotic cells express a CD4 peptide chain or CD9 peptide chain.

14. The method of claim 13, wherein the eukaryotic cells are selected from the group consisting of peripheral blood mononuclear cells, HuT-78 cells, and THP-1 cells.

15. The method of claim 9, wherein the molecule comprises an antibody.

16. A method of detecting a molecule that decreases IL-16 biological activity in a sample, comprising the steps of:
a) providing a first population of eukaryotic cells surrounded by media and responsive to IL-16 biological activity in a first test sample;
b) measuring the amount of a phosphorylated STAT-6 proxy produced by the first population of eukaryotic cells; and
c) comparing the amount of a phosphorylated STAT-6 proxy produced by the first population of eukaryotic cells with the amount of a phosphorylated STAT-6 proxy produced by a positive control sample containing biologically active IL-16, wherein a smaller amount of a phosphorylated STAT-6 proxy produced by the first population of eukaryotic cells relative to the phosphorylated STAT-6 proxy level produced by the positive control sample indicates the presence of a molecule that decreases IL-16 biological activity in the test sample.

17. The method of claim 16, wherein the providing step further comprises providing a second population of eukaryotic cells surrounded by media and responsive to IL-16 biological activity to form a positive control sample containing biologically active IL-16.

18. The method of claim 17, wherein the measuring step further comprises measuring the amount of a phosphorylated STAT-6 proxy produced by the second population of eukaryotic cells.

19. The method of claim 16, wherein the positive control sample is a second population of eukaryotic cells.

20. The method of claim 16, wherein the eukaryotic cells express a CD4 peptide chain or CD9 peptide chain.

21. The method of claim 20, wherein the eukaryotic cells are selected from the group consisting of peripheral blood mononuclear cells, HuT-78 cells, and THP-1 cells.

22. The method of claim 16, wherein the molecule comprises an antibody.

23. A method of diagnosing the presence of, or susceptibility to, an IL-16-related disorder in a first subject, comprising the steps of:
a) providing a first population of eukaryotic cells from a first subject;
b) measuring the amount of a phosphorylated STAT-6 proxy in the first population of eukaryotic cells; and
c) comparing the amount of a phosphorylated STAT-6 proxy in the first population of eukaryotic cells to the amount of a phosphorylated STAT-6 proxy in a reference sample, wherein a larger amount of a phosphorylated STAT-6 proxy in the first population of eukaryotic cells relative to the phosphorylated STAT-6 proxy level in the reference sample indicates the presence of, or susceptibility to, an IL-16-related disorder.

24. The method of claim 23, wherein the reference samples is selected from the group consisting of cells from a second subject not susceptible to an IL-16 related disorder, cells from a second subject not afflicted with an IL-16 related disorder, and cells not characterized by increased IL-16 expression or increased IL-16 biological activity.

25. The method of claim 23, wherein the providing step further comprises providing a second population of eukaryotic cells comprising cells selected from the group consisting of cells from a second subject not susceptible to an IL-16 related disorder, cells from a second subject not afflicted with an IL-16 related disorder, and cells not characterized by increased IL-16 expression or increased IL-16 biological activity to form the reference sample.
